(19) United States
(12) Patent Application Publication
(10) Pub. No.: US 2004/0248925 A1
(43) Pub. Date: Dec. 9, 2004

(54) Use of 4-phenyl substituted tetrahydroisoquinolines in the treatment of pain, migraine and urinary incontinence

(76) Inventors: Donald E. Frail, Wildwood, MO (US); Stephen P. Arneric, Milan, MI (US); Donn Gregory Wishka, Mystic, CT (US); Erik Ho Fong Wong, Portage, MI (US); James P. Beck, Zionsville, IN (US)

Correspondence Address: WARNER-LAMBERT COMPANY, 2800 PLYMOUTH RD, ANN ARBOR, MI, 48105, US

(21) Appl. No.: 10/725,221

(22) Filed: Dec. 1, 2003

Related U.S. Application Data
(60) Provisional application No. 60/430,298, filed on Dec. 2, 2002.

(51) Int. Cl. A61K 31/4745

(52) U.S. Cl. 514/291

(57) ABSTRACT

Provided herein are methods of using compounds of formula (IA, IB, IIA, IIB, IIIA, IIIB) that are aryl- and heteroaryl-substituted tetrahydroisoquinolines, for the treatment of chronic and neuropathic pain, the treatment and prevention of migraine headache, and the treatment of stress, urge and mixed urinary incontinence.

### CROSS REFERENCE

This application claims the benefit of the following provisional application: U.S. Ser. No. 60/430,298 filed Dec. 2, 2002 under 35 USC 119(e)(i), which is incorporated herein by reference in its entirety

### FIELD OF THE INVENTION

The present invention relates to methods for the treatment of various disorders. In particular, the present invention relates to methods wherein the compounds are 4-phenyl substituted tetrahydroisoquinoline derivatives.

### SUMMARY OF THE INVENTION

This invention provides various therapeutic uses of compounds of the Formulae IA, IB, IIA, IIB, IIIA and IIIB, as follows:

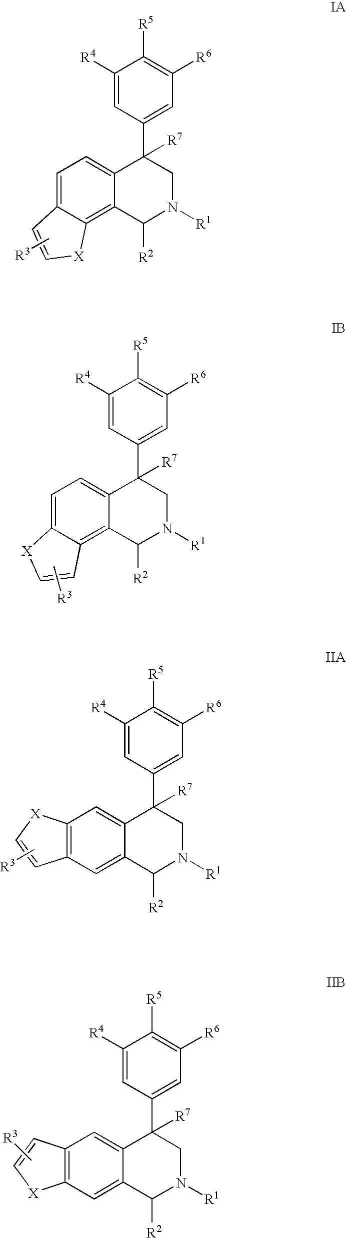

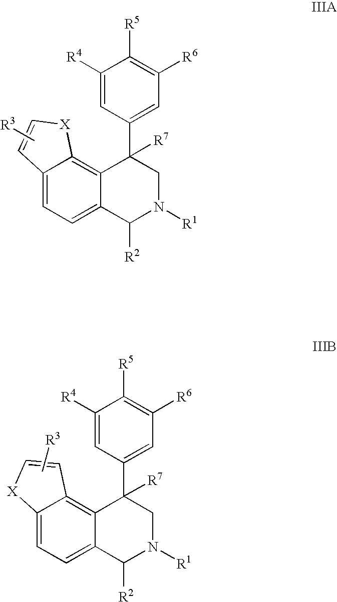

wherein R¹-R¹³ are as described herein. In one embodiment, R¹ is C₁-C₆ alkyl; R² is H, C₁-C₆ alkyl, C₃-C₆ cycloalkyl, or C₁-C₆ haloalkyl; R³ is at each occurrence thereof independently H, halogen, C₁-C₆ alkyl, or C₁-C₆ alkyl substituted with from 1 to 3 of OR⁸ or NR⁸R⁹; R⁴, R⁵ and R⁶ are each independently H or are selected at each occurrence thereof from halogen, OR¹⁰, NR¹⁰R¹¹, —NR¹⁰C(O)R¹¹, —S(O)ₙR¹¹, —CN, —C(O)(R¹¹, —C(O)₂R¹¹, C(O)NR¹¹R¹², C₁-C₆ alkyl, C₃-C₆ cycloalkyl, or C₄-C₇ cycloalkylalkyl, and wherein each of C₁-C₆ alkyl C₃-C₆ cycloalkyl, and C₄-C₇ cycloalkylalkyl is optionally substituted with from 1 to 3 substituents independently selected at each occurrence thereof from C₁-C₃ alkyl, halogen, —CN, —OR⁸, —NR⁸R⁹ and phenyl which is optionally substituted 1-3 times with halogen, —CN, C₁-C₄ alkyl, C₁-C₄ haloalkyl, —OR⁸, or —NR⁸R⁹; or R⁵ and R⁶ may be —O—C(R¹¹)₂—O—; and, R⁷-R¹³, n, and X are as described herein.

Compounds provided herein block the reuptake of norepinephrine, dopamine, and serotonin with particular selectivity ratios, e.g., being more selective for the norepinephrine transporter (NET) protein than the dopamine transporter (DAT) protein or serotonin transporter (SERT) proteins. Applicant has discovered that such compounds can be useful to treat chronic and neuropathic pain, to treat and prevent migraine headache, and to treat urge, stress and mixed urinary incontinence.

### DETAILED DESCRIPTION OF THE INVENTION

This invention provides the use of compounds of the Formulae IA, IB, IIA, IIB, IIIA or IIIB, to a) treat chronic and neuropathic pain, b) treat and prevent migraine headaches, and c) treat urge, stress and mixed urinary incontinence:

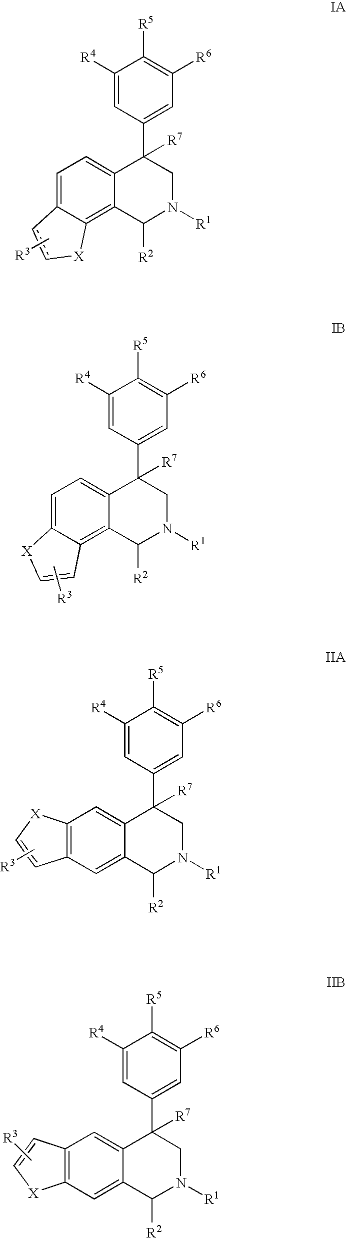

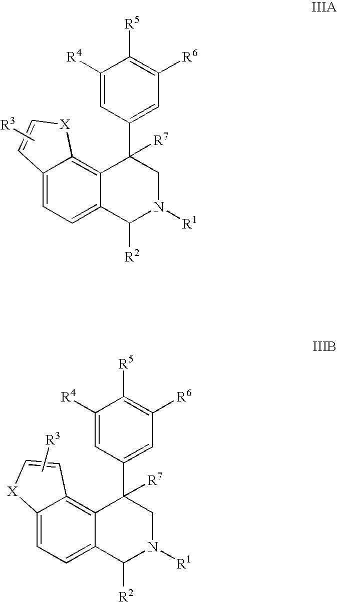

wherein:

R¹ is selected from the group consisting of C₁-C₆ alkyl, C₂-C₆ alkenyl, C₂-C₆ alkynyl, C₃-C₆ cycloalkyl, C₄-C₇ cycloalkylalkyl and benzyl, each of which is optionally substituted with 1 to 3 substituents independently selected at each occurrence from C₁-C3 alkyl, halogen, —CN, —OR⁸ and —NR⁸R⁹;

R² is selected from the group consisting of H, C₁-C₆ alkyl, C₂-C₆ alkenyl, C₂-C₆ alkynyl, C₃-C₆ cycloalkyl, C₄-C₇ cycloalkylalkyl and C₁-C₆ haloalkyl;

R³ is selected from the group consisting of H, halogen, C₁-C₆ alkyl, C₁-C₆ haloalkyl and C₃-C₆ cycloalkyl, wherein C₁-C₆ alkyl, C₁-C₆ haloalkyl and C₃-C₆ cycloalkyl are optionally substituted with 1 to 3 substituents selected independently at each occurrence from OR⁸ and NR⁸R⁹;

R⁴, R⁵, and R⁶ are each independently selected at each occurrence thereof from the group consisting of H, halogen, —OR¹⁰, —NO₂, —NR¹⁰R¹¹, —NR¹⁰C(O)R¹¹, —NR¹⁰C(O)NR¹¹R¹², —S(O)ₙR¹¹, —CN, —C(O)R¹¹, —C(O)₂R¹¹, —C(O)NR¹¹R¹², C₁-C₆ alkyl, C₂-C₆ alkenyl, C₂-C₆ alkynyl, C₃-C₆ cycloalkyl and C₄-C₇ cycloalkylalkyl, wherein each of C₁-C₆ alkyl, C₂-C₆ alkenyl, C₂-C₆ alkynyl, C₃-C₆ cycloalkyl and C₄-C₇ cycloalkylalkyl are optionally substituted with 1 to 3 substituents independently selected at each occurrence with from C₁-C₃ alkyl, halogen, =O, —CN, —OR⁸, —NR⁸R⁹ and phenyl, and wherein phenyl is optionally substituted 1-3 substituents selected independently at each occurrence from halogen, —CN, C₁-C₄ alkyl, C₁-C₄ haloalkyl, —OR⁸ and —NR⁸R⁹;

alternatively R⁵ and R⁶ are —O—C(R¹¹)₂—O—;

R⁷ is selected from the group consisting of H, halogen and OR¹⁰;

R⁸ and R⁹ are each independently selected from the group consisting of H, C₁-C₄ alkyl, C₁-C₄ haloalkyl, C₁-C₄ alkoxyalkyl, C₁-C₄ alkoxyalkylalkyl, C₃-C₆ cycloalkyl, C₄-C₇ cyclooalkylalkyl, —C(O)R¹², phenyl and benzyl, wherein phenyl and benzyl are optionally substituted with 1 to 3 substituents selected independently at each occurrence from halogen, cyano, C₁-C₄ alkyl, C₁-C₄ haloalkyl, C₁-C₄ alkoxy and C₁-C₄ haloalkoxy, or R¹ and R⁹ are taken together with the nitrogen to which they are attached to form a piperidine, pyrrolidine, piperazine, N-methylpiperazine, morpholine, or thiomorpholine ring;

R¹⁰ is selected from the group consisting of H, C₁-C₄ alkyl, C₁-C₄ haloalkyl, C₁-C₄ alkoxyalkyl, C₃-C₆ cycloalkyl, C₄-C₇ cycloalkylalkyl, —C(O)R¹², phenyl and benzyl, wherein phenyl and benzyl are optionally substituted with 1 to 3 substituents selected independently at each occurrence from halogen, —NH₂, —OH, cyano, C₁-C₄ alkyl, C₁-C₄ haloalkyl, C₁-C₄ alkoxy and C₁-C₄ haloalkoxy;

R¹¹ is selected from the group consisting of H, C₁-C₄ alkyl, C₁-C₄ haloalkyl, C₁-C₄ alkoxyalkyl, C₃-C₆ cycloalkyl, C₄-C₇ cycloalkylalkyl, phenyl and benzyl, where phenyl and benzyl are optionally substituted with 1 to 3 substituents selected independently at each occurrence from halogen, —NH₂, —OH, cyano, C₁-C₄ alkyl, C₁-C₄ haloalkyl, C₁-C₄ alkoxy and C₁-C₄ haloalkoxy, or R¹⁰ and R¹¹ are taken together with the nitrogen to which they are attached to form a piperidine, pyrrolidine, N-methylpiperazine, morpholine, or thiomorpholine ring, with the proviso that only one of R⁸ and R9 or R¹⁰ and R¹¹ are taken together with the nitrogen to which they are attached to form a piperidine, pyrrolidine, piperaine, N-methylpiperazine, morpholine, or thiomorpholine ring;

R¹² is selected from the group consisting of C₁-C₄ alkyl, C₁-C₄ haloalkyl and phenyl;

X is selected from the group consisting of O, NR¹³ and S, with the proviso that X is not NR¹³ when a compound is of Formula (IA);

the ring containing X is selected from furan, pyrrole, thiophene, dihydrofuran, dihydropyrrole, and dihydrothiophene; n is 0, 1, or 2; and,

R¹³ is selected from the group consisting of H, C₁-C₆ alkyl, benzyl and phenyl, wherein C₁-C₆ alkyl, benzyl and phenyl are optionally substituted with 1-3 substituents independently at each occurrence from halogen, —NH₂, —OH, cyano, C₁-C₄ alkyl, C₁-C₄ haloalkyl, C₁-C₄ alkoxy and C₁-C₄ haloalkoxy.

“Alkyl” means saturated hydrocarbon chains, branched or unbranched, having the specified number of carbon atoms. “Alkenyl” means hydrocarbon chains of either a straight or branched configuration and one or more unsaturated carbon-carbon bonds, which may occur in any stable point along the chain, such as ethenyl, propenyl, and the like. “Alkynyl” means hydrocarbon chains of either a straight or branched configuration and one or more triple carbon-carbon bonds, which may occur in any stable point along the chain, such as ethynyl, propynyl, and the like. “Alkoxy” means an alkyl group of the indicated number of carbon atoms attached through an oxygen bridge. “Cycloalkyl” means saturated ring groups, including mono-, bi-, or poly-cyclic ring systems, such as cyclopropyl, cyclobutyl, cyclopentyl, and the so forth. “Halo” or “halogen” means fluoro, chloro, bromo, and iodo. “Haloalkyl” means both branched and straight-chain alkyls having the specified number of carbon atoms, substituted with 1 or more halogen. “Haloalkoxy” means an alkoxy group substituted by at least one halogen atom.

“Substituted” or “substitution” of an atom means that one or more hydrogen on the designated atom is replaced with a selection from the indicated group, provided that the designated atom's normal valence is not exceeded. “Unsubstituted” atoms bear all of the hydrogen atoms dictated by their valency. When a substituent is keto (ie. C═O), then 2 hydrogens on the atom are replaced. Combinations of substituents and/or variables are permissible only if such combinations result in stable ‘compounds; by “stable compound” or “stable structure” is meant a compound that is sufficiently robust to survive isolation to a useful degree of purity from a reaction mixture, and formulation into an efficacious therapeutic agent.

One embodiment of this invention are those compounds wherein: R¹ is C₁-C₆ alkyl; R² is H, C₁-C₆ alkyl, C₃-C₆ cycloalkyl, or C₁-C₆ haloalkyl; R³ is at each occurrence thereof independently H, halogen, C₁-C₆ alkyl, or C₁-C₆ alkyl substituted with from 1 to 3 of OR⁸ or NR⁸R⁹; R⁴, R⁵ and R⁶ are each independently H or are selected at each occurrence thereof from halogen, —OR¹⁰, —NR¹⁰R¹¹, —NR¹⁰C(O)R¹¹, —S(O)ₙR¹¹, —CN, —C(O)R¹¹, —C(O)₂R¹¹, —C(O)NR¹¹R¹², C₁-C₆ alkyl, C₃-C₆ cycloalkyl, or C₄-C₇ cycloalkylalkyl, and wherein each of C₁-C₆ alkyl, C₃-C₆ cycloalkyl, and C₄-C₇ cycloalkylalkyl is optionally substituted with from 1 to 3 substituents independently selected at each occurrence thereof from C₁-C₃ alkyl, halogen, —CN, —OR⁸, —NR⁸R⁹ and phenyl which is optionally substituted 1-3 times with halogen, —CN, C₁-C₄ alkyl, C₁-C₄ haloalkyl, —OR⁸, or —NR⁸R⁹; or R⁵ and R⁶ may be —O—C(R¹¹)₂—O—; and, R⁷—R¹³, n, and X are described above.

Within these embodiments, the selection of a particular substituent on any one position of a compound does not necessarily affect the selection of a substituent at another position on the same compound—that is, compounds provided herein have any of the substituents at any of the positions. For example, as described hereinabove, R¹ is preferably, for example, C₁-C₆ alkyl—the selection of R¹ as any one of C₁, C₂, C₃, C₄, C₅, or C₆ alkyl, does not limit the choice of R² in particular to any one of H, C₁-C₆ alkyl, C₃-C₆ cycloalkyl, or C₁-C₆ haloalkyl. Rather, for R¹ as any of C₁, C₂, C₃, C₄, C₅, or C₆ alkyl, R² is any of H, C₁, C₂, C₃, C₄, C₅, or C₆ alkyl or C₃, C₄, C₅, or C₆ cylcoalkyl, or C₁, C₂, C₃, C₄, C₅, or C₆ haloalkyl. Similarly, the selection of R² in particular to any one of H, C₁, C₂, C₃, C₄, C₅, or C₆ alkyl or C₃, C₄, C₅, or C₆ cylcoalkyl, or C₁, C₂, C₃, C₄, C₅, or C₆ haloalkyl does not limit the selection of R³ in particular to any one of its constituent members.

In another embodiment, R¹ is methyl, ethyl, propyl or isopropyl; R² is H or C₁-C₆ alkyl, and R³ is H, halogen, or C₁-C₆ alkyl, wherein C₁-C₆ alkyl is optionally substituted with from 1-30R⁸; R⁴ and R⁵ and R⁶ are each independently H, halogen, —OR¹⁰, —S(O)ₙR¹¹—, —NR¹⁰R¹¹, —C(O)R¹¹, or C₁-C₆ alkyl wherein C₁-C₆ alkyl is optionally substituted as described above; and R⁷-R¹³ and X are as described above. In yet another embodiment, R¹ is methyl; R² and R³ are H; R⁴ and R⁵ and R⁶ are each independently H, F, Cl, —OH, C₁-C₃ alkoxy, or C₁-C₃ alkyl; R⁷ is H, F, —OH, or —OCH₃ and; R⁸-R¹³ and X are as described above.

In one embodiment compounds include, for example and without limitation, those compounds set forth in Tables I-VIA herein below. That is, such compounds include those having the following formula:

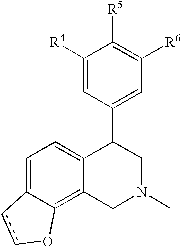

wherein the oxygen-containing ring is either saturated or unsaturated, R⁴ is H, Cl or F, R⁵ is H, F or Me and R⁶ is H or F. In another embodiment compounds include those having the following formula:

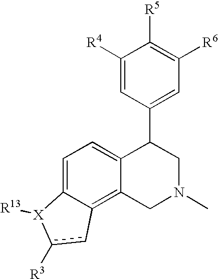

wherein X is O, S or N, the X-containing ring is either saturated or unsaturated, R³ is H, Me, Et or MeOH, R⁴ and R⁶ are each H, F or Cl, R⁵ is H, F, Cl or OMe and R¹³ when present, is C₁-C₆ alkyl. Yet in another embodiment compounds further include those having the following formula:

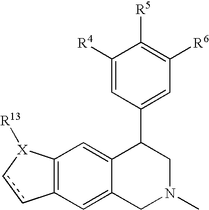

wherein X is O or N, the X-containing ring is either saturated or unsaturated, R⁴, R⁵ and R⁶ are each H and R¹³ when present, is H or C₁-C₆ alkyl.

Still another embodiment includes compounds having the following formula:

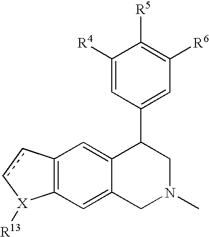

wherein X is O or N, the X-containing ring is either saturated or unsaturated, R⁴ is H, R⁵ is H, Cl, F or Br, R⁶ is H, Cl or F and R¹³ is H or C₁-C₆ alkyl. Further embodiments include those compounds having the following formula:

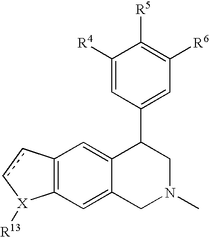

wherein X is O or S, the X-containing ring is either saturated or unsaturated, R⁴ is H, R⁵ is H, Cl, F or OMe, R⁶ is H, Cl or F and R¹³ is C₁-C₆ alkyl. In yet another embodiment compounds include those having the following formula:

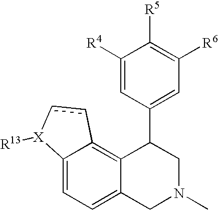

wherein X is 0, the X-containing ring is either saturated or unsaturated, R⁴ is H, R⁵ is H or F, and R⁶ is H or F.

Each of the stereoisomeric forms of this invention's compounds is also provided for herein. That is, the compounds can have one or more asymmetric centers or planes, and all chiral (enantiomeric and diastereomeric) and racemic forms of the compounds are included in the present invention. Many geometric isomers of olefins, C═N double bonds, and the like can also be present in the compounds, and all such stable isomers are contemplated in the present invention. Compounds are isolated in either the racemic form, or in the optically pure form, for example, by chiral chromatography or chemical resolution of the racemic form.

Pharmaceutically acceptable salts of this invention's compounds are also provided for herein. In this regard, the phrase “pharmaceutically acceptable” is employed to refer to those compounds, materials, compositions, and/or dosage forms that are within the scope of sound medical judgment, suitable for use in contact with the tissues of human beings and animals without excessive toxicity, irritation, allergic response or other problem or complication, commensurate with a reasonable benefit/risk ratio.

“Pharmaceutically acceptable salts” refers to derivatives of the disclosed compounds wherein the parent compound is modified by making acid or base salts thereof.

Examples of pharmaceutically acceptable salts include, but are not limited to, mineral or organic acid salts of basic residues such as amines; alkali or organic salts of acidic residues such as carboxylic acids; and the like. Pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

Prodrug forms of this invention's compounds are also provided for herein. Such “prodrugs” are compounds comprising this invention's compounds and moieties covalently bound to the parent compounds such that the portions of the parent compound most likely to be involved with toxicities in subjects to which the prodrugs have been administered are blocked from inducing such effects. However, the prodrugs are also cleaved in the subjects in such a way as to release the parent compound without unduly lessening its therapeutic potential. Prodrugs include compounds wherein hydroxy, amine, or sulfhydryl groups are bonded to any group that, when administered to a mammalian subject, cleaves to form a free hydroxyl, amino, or sulfhydryl group, respectively. Examples of prodrugs include, but are not limited to, acetate, formate, and benzoate derivatives of alcohol, and amine functional groups in the compounds of Formulae (I-III).

Radiolabelled compounds, i.e., wherein one or more of the atoms described are replaced by a radioactive isotope of that atom (e.g., C replaced by ¹⁴C or by ¹¹C, and H replaced by ³H or ¹⁸F), are also provided for herein. Such compounds have a variety of potential uses, e.g., as standards and reagents in determining the ability of a potential pharmaceutical to bind to neurotransmitter proteins, or for imaging compounds of this invention bound to biological receptors in vivo or in vitro.

“Therapeutically effective amounts” are any amounts of the compounds effective to ameliorate, lessen, inhibit or prevent the particular condition for which a subject is being treated. Such amounts generally vary according to a number of factors well within the purview of ordinarily skilled artisans given the description provided herein to determine and account for. These include, without limitation: the particular subject, as well as its age, weight, height, general physical condition and medical history; the particular compound used, as well as the carrier in which it is formulated and the route of administration selected for it; and, the nature and severity of the condition being treated. Therapeutically effective amounts include optimal and suboptimal doses, and can be determined in a variety of ways known to ordinarily skilled artisans, e.g., by administering various amounts of a particular agent to an animal afflicted with a particular condition and then determining the relative therapeutic benefit received by the animal. The amounts generally range from about 0.001 mg per kg of the body weight of the subject being treated to about 1000 mg per kg, and more typically, from about 0.1 to about 200 mg per kg. These amounts can be administered according to any dosing regimen acceptable to ordinarily skilled artisans supervising the treatment. More specific doses are mentioned below in relationship to the treatment of particular disorders that are the subject of this invention.

“Pharmaceutically acceptable carriers” are media generally accepted in the art for the administration of therapeutic compounds to humans. Such carriers are generally formulated according to a number of factors well within the purview of those of ordinary skill in the art to determine and account for. These include, without limitation: the type and nature of the active agent being formulated; the subject to which the agent-containing composition is to be administered; the intended route of administration of the composition; and, the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, well known to those of ordinary skill in the art. Descriptions of suitable pharmaceutically acceptable carriers, and factors involved in their selection, are found in a variety of readily available sources, e.g., Remington's Pharmaceutical Sciences, 17^{th} ed., Mack Publishing Company, Easton, Pa., 1985, the contents of which are incorporated herein by reference.

Compounds of this invention are administered, for example, parenterally in various aqueous media such as aqueous dextrose and saline solutions; glycol solutions are also useful carriers. Solutions for parenteral administration preferably contain a water soluble salt of the active ingredient, suitable stabilizing agents, and if necessary, buffer substances. Antioxidizing agents, such as sodium bisulfite, sodium sulfite, or ascorbic acid, either alone or in combination, are suitable stabilizing agents. Also used are citric acid and its salts, and EDTA. In addition, parenteral solutions can contain preservatives such as benzalkonium chloride, methyl- or propylparaben, and chlorobutanol.

Alternatively, the compounds are administered orally in solid dosage forms, such as capsules, tablets and powders, or in liquid forms such as elixirs, syrups, and/or suspensions. Gelatin capsules can be used to contain the active ingredient and a suitable carrier such as but not limited to lactose, starch, magnesium stearate, steric acid, or cellulose derivatives. Similar diluents can be used to make compressed tablets. Both tablets and capsules can be manufactured as sustained release products, to provide for continuous release of medication over a period of time. Compressed tablets can be sugarcoated or film-coated to mask any unpleasant taste, or used to protect the active ingredients from the atmosphere, or to allow selective disintegration of the tablet in the gastrointestinal tract.

Compounds of this invention provide a particularly beneficial therapeutic index relative to other compounds available for the treatment of similar disorders. Without intending to be limited by theory, it is believed that this is due, at least in part, to the compounds, ability to be selective for the norepinephrine transporter protein (NET) over the other neurotransmitter transporters. Binding affinities are demonstrated by a number of means well known to ordinarily skilled artisans.

Briefly, for example, protein containing extracts from cells, e.g., HEK293 cells, expressing the transporter proteins are incubated with radiolabelled ligands for the proteins. The binding of the radioligands to the proteins is reversible in the presence of other protein ligands, e.g., the compounds of this invention; said reversibility, as described below, provides a means of measuring the compounds' binding affinities for the proteins (Ki). A higher Ki value f or a compound is indicative that the compound has less binding affinity for a protein than is so for a compound with a lower Ki; conversely, lower Ki values are indicative of greater binding affinities.

Accordingly, a lower Ki for the protein for which the compound is more selective, and a higher Ki for the protein for which the compound is less selective indicate the difference in compound selectivity for proteins. Thus, the higher the ratio in Ki values of a compound for protein A over protein B, the greater is the compounds' selectivity for the latter over the former (the former having a higher Ki and the latter a lower Ki for that compound). Compounds provided herein induce fewer side effects during therapeutic usage because of their selectivity for the norepinephrine transporter protein, as indicated by the ratios of their Ki's for binding to NET over those for binding to other transporter proteins, e.g., the dopamine transporter (DAT) and the serotonin transporter (SERT). Generally, the compounds of this invention have a Ki ratio for DAT/NET of about ≧2:1; the compounds generally also have a SERT/NET ratio of about ≧5:1.

Moreover, in vivo assessment of the activity of compounds at the NE and DA transporters is, for example, by determining their ability to prevent the sedative effects of tetrabenazine (TBZ) (see, e.g., G. Stille, Arzn. Forsch. 1964, 14, 534-537; the contents of which are incorporated herein by reference). Randomized and coded doses of test compounds are administered to mice, as is then a dose of tetrabenazine. Animals are then evaluated for antagonism of tetrabenazine-induced exploratory loss and ptosis at specified time intervals after drug administration. Exploratory activity is, for example, evaluated by placing the animal in the center of a circle and then evaluating the amount of time it takes for the animal to intersect the circle's perimeter—generally, the longer it takes for the animal to make this intersection, the greater is its loss of exploratory activity. Furthermore, an animal is considered to have ptosis if its eyelids are at least 50% closed. Greater than 95% of the control (vehicle-treated) mice are expected to exhibit exploratory loss and ptosis; compound-related activity is then calculated as the percentage of mice failing to respond to the tetrabenazine challenge dose, with therapeutically more effective compounds expected to be better at reducing loss of exploratory behavior and ptosis.

Accordingly, the pharmaceutical compositions provided herein are useful in the treatment of subjects afflicted with various neurological and psychiatric disorders by administering to said subjects a dose of a pharmaceutical composition provided herein. Said disorders include, without limitation, chronic and neuropathic pain, migraine therapy and prevention, and urge, stress and mixed urinary incontinence. The compounds provided herein, are particularly useful in the treatment of these and other disorders due, at least in part, to their ability to selectively bind to the transporter proteins for certain neurochemicals with a greater affinity than to the transporter proteins for other neurochemicals.

The compounds of the present invention can be prepared using the methods described in International Application WO 02/04455, together with methods known in the art of synthetic organic chemistry, or variations thereof as appreciated by those skilled in the art.

In order to evaluate the relative affinity of the various compounds at the NE, DA and 5HT transporters, HEK293E cell lines can be developed to express each of the three human transporters. cDNAs containing the complete coding regions of each transporter can be amplified by PCR from human brain libraries. The cDNAs contained in pCRII vectors can be sequenced to verify their identity and then subcloned into an Epstein Barr virus based expression plasmid (E. Shen, G M Cooke, R A Horlick, Gene 156:235-239, 1995). This plasmid containing the coding sequence for one of the human transporters can be transfected into HEK293E cells. Successful transfection can be verified by the ability of known reuptake blockers to inhibit the uptake of tritiated NE, DA or 5HT.

For binding, cells can be homogenized, centrifuged and then resuspended in incubation buffer (50 mM Tris, 120 mM NaCl, 5 mM KCl, pH 7.4). Then the appropriate radioligand can be added. For NET binding, [³H] Nisoxetine (86.0 Ci/mmol, NEN/DuPont) can be added to a final concentration of approximately 5 nM. For DAT binding, [³H] WIN 35,428 (84.5 Ci/mmol) at 15 nM was added. For 5HTT binding, [³H] Citolapram (85.0 Ci/mmol) at 1 nM was added. Then various concentrations (10-5 to IOA-11 M) of the compound of interest can be added to displace the radioligand. Incubation can be carried out at room temperature for 1 hour in a 96 well plate. Following incubation, the plates can be placed on a harvester and washed quickly 4 times with (50 mM tris, 0.9% NaCl, pH 7.4) where the cell membranes containing the bound radioactive label can be trapped on Whatman GF/B filters. Scintillation cocktail can be added to the filters that were then counted in a Packard TopCount. Binding affinities of the compounds of interest can be determined by non-linear curve regression using GraphPad Prism 2.01 software. Non-specific binding can be determined by displacement with 10 micromolar mazindol.

In order to assess in vivo activity of the compounds at the NE and DA transporters, their ability to prevent the sedative effects of tetrabenazine (TBZ) can be determined (G. Stille, Arzn. Forsch 14:534-537, 1964). Male CFI mice (Charles River Breeding Laboratories) weighing 18-25 gm at the time of testing, can be housed a minimum of 6 days under carefully controlled environmental conditions (22.2+1.1 C; 50% average humidity; 12 hr lighting cycle/24 hr). Mice can be fasted overnight (16-22 hr) prior to testing. Mice can be placed into clear polycarbonated “shoe” boxes (17 cm×28.5 cm×12 cm).

Randomized and coded doses of test compounds can be administered p.o. A 45 mg/kg dose of tetrabenazine can be administered i.p. 30 minutes prior to score time. All compounds can be administered in a volume of 0.1 ml/10 gm body weight. Animals can be evaluated for antagonism of tetrabenazine induced exploratory loss and ptosis at specified time intervals after drug administration. At the designated time interval, mice are examined for signs of exploratory activity and ptosis. Exploratory activity can be evaluated by placing the animal in the center of a 5-inch circle. Fifteen seconds can be allowed for the animal to move and intersect the perimeter. This can be considered antagonism of tetrabenazine and given a score of 0. Failure to leave the circle can be regarded as exploratory loss and given a score of 4. An animal can be considered to have ptosis if its eyelids are at least 50% closed and can be given a score of 4 if completely closed; no closure can be given a score of 0. Greater than 95% of the control (vehicle-treated) mice can be expected to exhibit exploratory loss and ptosis. Drug activity can be calculated as the percentage of mice failing to respond to the tetrabenazine challenge dose.

Median effective doses (ED50s) and 95% confidence limits 30 can be determined numerically by the methods of Thompson (1947) and Litchfield and Wilcoxon (1949).

Chronic painful conditions, in various forms, affect a considerable number of people including, according to the WHO, 4 million cancer sufferers who, worldwide, suffer as a result of a lack of suitable care. There are a number of other conditions, such as musculoskeletal or vertebral pain, neurological pain, headaches or vascular pain. Neurophathic pain, a chronic pain condition occurring in the setting of nervous system injury or tissue injury, is characterized by unusual sensory experiences (allodynia, hyperalgesia) and abnormal pain processing in the central and peripheral nervous systems; treatment of neuropathic pain is difficult. Painful diabetic neuropathy is one of the most frequent complications of diabetes in humans, post-herpetic neuralgia develops in 10-30% of patients after herpes zoster, phantom limb and stump pain is a common sequela of amputation. Chronic pain may also be caused by a trauma, an entrapment neuropathy (e.g., carpal tunnel syndrome), multiple sclerosis or a polyneurophathy associated with AIDS, alcoholism, hypothyroidism, or anticancer chemotherapy.

Conventional treatments of pain fall into two categories: 1) nonsteroidal anti-inflammatory drugs (NSAIDs), used to treat mild pain, but whose therapeutic use is limited by GI adverse effects; and 2) morphine and related opiods, used to treat moderate to severe pain but whose therapeutic use is limited by undesirable side effects including respiratory depression, tolerance, and abuse potential. However, conventional analgesics, whether opiates or NSAIDs, have limited therapeutic value in the management of chronic pain syndromes. This has led to the use of adjuvant analgesics for the management of these conditions. For example, tricyclic antidepressants are currently the first choice in the treatment of painful diabetic neuropathy. However, few agents are fully effective in all patients and undesirable side effects are common.

For use in the treatment of chronic pain or neuropathic pain the compounds of formula IA, IB, IIA, IIB, IIIA, and IIIB may be administered orally or parenterally in an amount sufficient to alleviate the symptoms of chronic pain or neuropathic pain. The actual amount of a compound of formula I to be used will vary with the severity and nature of the state of chronic or neuropathic pain, the animal being treated and the level of relief sought. In the human, an oral dose of from about 2 to about 50 milligrams, administered as needed represents appropriate posology. Intramuscular administration of from about 1 to about 25 milligrams provides a dosage comparable to that specified for oral administration.

As used herein the term “chronic pain” means pain selected from causalgia, neuropathic pain, diabetic neuropathy, post-surgery or traumatic neuropathy, postherpetic neuralgia, peripheral neuropathy, entrapment neuropathy, phantom limb and stump pain, neuropathy caused by alcohol abuse, HIV infection, multiple sclerosis, hypothyroidism, lower back pain, cancer pain and pain from anticancer chemotherapy. Applicant particularly prefers the use of the compounds of formula IA, IB, IIA, IIB, IIIA, and IIIB for the treatment of neuropathic pain.

The term “chronic pain relieving amount” represents an amount of a compound of formula IA, IB, IIA, IIB, IIIA, and IIIB which is capable of relieving or reducing chronic pain in a mammal in need thereof.

The pain of migraine is associated with excessive dilatation of the cranial vasculature and known treatments for migraine include the administration of compounds having vasoconstrictor properties such as ergotamine. However, ergotamine is a non-selective vasoconstrictor that constricts blood vessels throughout the body and has undesirable and potentially dangerous side effects. Migraine may also be treated by administering an analgesic, usually in combination with an antiemetic, but such treatments are of limited value.

There is thus a need for a safe and effective drug for the treatment of migraine, which can be used either prophylactically or to alleviate an established headache, and a compound having a selective vasoconstrictor activity would fulfill such a role.

Furthermore, in conditions such as migraine, where the drug will usually be administered by the patient, it is highly desirable that the drug can be taken orally. It should therefore possess good bioavailability and be effectively absorbed from the gastrointestinal tract so that prompt relief of symptoms can occur. The drug should also be safe (i.e., free from toxic effects) when administered by the oral route.

It is generally believed that the pain of migraine is of vascular origin and caused by excessive dilation of branches of the common carotid arterial bed. (J. W. Lance, Mechanisms and Management of Migraine, Butterworths, p 113-152 (1973). The role of norepinephrine reuptake in the management of migraine headache pain is discussed in J. R. Couch, et al., Amitriptyline in the prophylaxis of migraine, Neurology 1976:26:121-127 and S. Diamond, et al., Chronic tension headache treated with amitruptyline: a double blind study, Headache 1971; 11:110-116.

A proposed dose of the compounds of the invention for oral administration to man (about 70 kg bodyweight) for the treatment of migraine is 0.1 mg to 100 mg, for example, 0.5 mg to 50 mg, preferably 2 mg to 40 mg, of the active ingredient per dose which could be administered up to 4 times per day, more usually 1 to 2 times per day. It will be appreciated that it may be necessary to make routine variations to the dosage depending on the age and weight of the patient, as well as the severity of the condition to be treated. It should be understood that unless otherwise indicated, the dosages are referred to in terms of the weight of compound (I) as the free base.

According to a further aspect, the invention provides a method of treatment of a human subject suffering from or susceptible to pain resulting from dilatation of the cranial vasculature, such as migraine or cluster headache, by administration of a compound of formula (I) or a physiologically acceptable salt or solvate thereof. The method of treatment preferably comprises oral administration of a compound of the invention.

Urinary incontinence is generally defined as the involuntary loss of urine and is most common in four groups of patients including children, women, elderly, and neurologic disease patients. Detrusor instability is characterized by spasmodic bladder contractions or bladder contractions elicited by small volumes and is often accompanied by incontinence and urinary frequency. Interstitial cystitis is an idiopathic pelvic pain syndrome that can also include detrusor instability as a component of its pathology.

Nocturnal enuresis is classified as an involuntary micturition during sleep after 5 years of age and may exist in either primary or secondary forms. The diagnosis of primary nocturnal enuresis is made if the patient has never developed voluntary control of micturition during sleep. The diagnosis of secondary nocturnal enuresis is made if the patient has had transient periods of micturition control during sleep. Nocturnal enuresis occurs in 30% of all children at 4 years of age, 10% at 6 years, 3% at 10 years and 1% at 18 years. Secondary nocturnal enuresis accounts for approximately 20-25% of the pediatric enurenic cases. Although some enuretic children also have diurnal enuresis, over 80% of the enuretic children have exclusively nocturnal enuresis.

The predominant types of incontinence in women are stress and urge incontinence. Stress incontinence is the involuntary loss of urine through an intact urethra produced during times of increased abdominal pressure such as during physical activity and coughing. This implies that the urethra cannot generate sufficient pressure for outlet resistance to compensate for increases in intrabladder pressure. This loss of urine is not accompanied by premonitory sensations of the need to void and is not related to the fullness of the bladder. Urge incontinence is the involuntary loss of urine through an intact urethra due to an increased intrabladder pressure. In contrast to stress incontinence, urge incontinence is caused by an episodic bladder contraction (detrusor instability) which exceeds the outlet resistance pressure generated by the urethra and is accompanied by a perception of urgency to void.

Stress incontinence is the most common form of incontinence in young women. In two longitudinal studies, pure stress incontinence was found to occur in 15-22% of women from ages 17-75+. The highest incidence of stress incontinence (25-30%) occurs at 25-45 years of age or during the childbearing years. Following the first child birth, the overall incidence and incidence of severe stress incontinence doubles. However, 35-50% of nulliparous women have also occasional stress incontinence. In a study of nulliparous nursing students between the ages of 17-24 years, daily stress incontinence was reported in 17% of the women. Urge incontinence occurs in approximately 10% of women from ages 17-75+years and increases progressively with age. In addition to stress or urge incontinence, 7-14% of women from ages 17-75+years of age have characteristics of both urge and stress incontinence. The incidence of this “complex incontinence” doubles during the childbearing years and ranges from 13-28% from ages 17 to 75+years of age.

The types of incontinence seen in the elderly include urge incontinence (detrusor instability), stress incontinence, complex incontinence (urge and stress incontinence) and total incontinence. Urge incontinence is the most common form of incontinence in the elderly men and women and is caused by abnormal neuromuscular responses of the bladder. Following urge incontinence in incidence are complex, stress, overflow and total incontinence, respectively. Stress incontinence is relatively rare in elderly men but common in women. Stress incontinence is caused by pelvic surgery, anatomical changes in the orientation of the bladder and urethra, decreased tone of the pelvic muscles, deterioration of the urethra following the cessation of estrogen secretion, and idiopathic decrease in the neuromuscular response of tile urethra. Overflow incontinence is due to an overfilling and distension of an areflexic bladder that exceeds the urethral resistance. Total incontinence is associated with dementia and sphincter or nerve damage.

In addition to the types of incontinence described above, urge incontinence is also associated with neurologic disorders such as multiple sclerosis, Alzheimer's disease and Parkinson's disease. This urge incontinence caused by neurologic disorders result from bladder hyperactivity. The incidence of incontinence in multiple sclerosis patients has been estimated to be 60-90%. Urinary incontinence is among the early neurologic symptoms of Parkinson's disease patients and is frequently exacerbated by treatment with anti-Parkinson drugs.

Interstitial cystitis is a syndrome that is characterized by increases in urination frequency, urgency, suprapubic pressure and pain with bladder filling. This syndrome is not associated with infections or cytological damage. The average age at onset of this disorder is 40-50 years. The quality of life is considered to be worse than that of end stage renal disease. According to the NIH report on interstitial cystitis, there are 20,000 to 90,000 diagnosed cases of this disorder in United States and the upper boundary for undiagnosed cases is 4-5 times larger than the range of diagnosed cases. This disorder has increased in awareness in the urologic community due to the formation of the American Interstitial Cystitis Association.

The treatments for incontinence vary with the particular type. For example, with no therapy, the spontaneous cure rate for nocturnal enuresis is approximately 15% per year. The success rate for nonpharmacologic therapies such as motivational counseling, bladder exercises and enuresis alarms ranges from 25-70%. The tricyclic antidepressants have been the most effective pharmacologic agents for treating nocturnal enuresis. Imipramine is the most widely used agent; however other tricyclics such as nortriptyline, amitriptyline, and desipramine are also effective. Enuresis can be cured in over 50% of patients following treatment with imipramine and improvements can be seen in another 15-20%. A successful response to this therapy is usually seen in the first week of therapy and often after the first dose. The best results are seen in children with normal sized bladders who are occasionally continent at night. The worst results are seen in children with small bladders and in older adolescents. This therapy, however, does have toxic risks. The tricyclic anti-depressants in general, and imipramine in particular, are not approved for use in children under 5 years of age as these compounds are particularly toxic and potentially lethal in low dosage. Other pharmacologic therapies include the use of oxybutynin, antispasmotic agent that reduces uninhibited detrusor muscles contractions, and the antidiuretic agent desmopressin.

The predominant forms of therapy for incontinent women include a variety of surgical procedures that attempt to resuspend the bladder and/or reinforce the urethra; pelvic floor exercises; and pharmacologic therapies. Imipramine is effective as a single therapy in restoring continence to women with stress incontinence. The efficacy of imipramine in urge incontinence has varied along clinical studies and appears greater when used as a combination therapy with anticholinergic and antispasmotic agents.

The amount of compound required to effectively treat incontinence will depend upon the compound employed and its relative potency for effecting monoamine reuptake inhibition. Such doses can be generally extrapolated based upon the in vitro and any in vivo testing such as that mentioned above. For example, for adult patients, a compound of this invention would be expected to be effective when administered in amounts of 20-200 milligrams per day. However, it should be readily understood that the amount of the compound actually administered will be determined by a physician, in light of all the relevant circumstances including the particular condition to be treated, the choice of compound to be administered, and the choice of route of administration.

What is claimed is:

1. A method of treating chronic or neuropathic pain, treating or preventing migraine headache, or treating urge, stress or nixed urinary incontinence comprising administration of an effective amount of a compound selected from one of the Formulae IA, IB, IIA, IIB, IIIA or IIIB 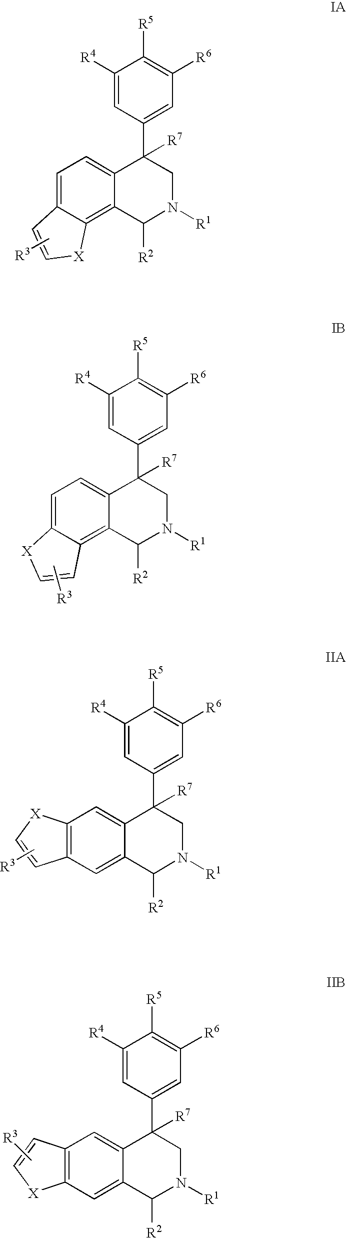 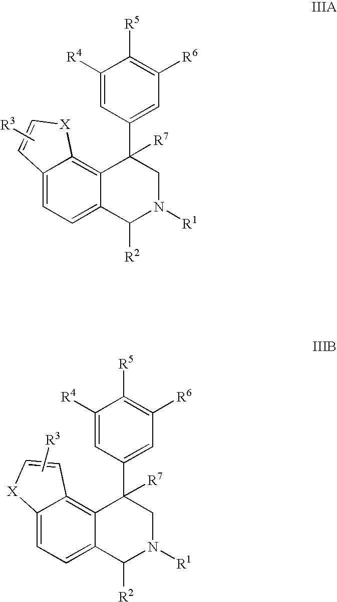
wherein:
R¹ is selected from the group consisting of C₁-C₆ alkyl, C₂-C₆ alkenyl, C₂-C₆ alkynyl, C₃-C₆ cycloalkyl, C₄-C₇ cycloalkylalkyl and benzyl, each of which is optionally substituted with 1 to 3 substituents independently selected at each occurrence from C₁-C₃ alkyl, halogen, —CN, —OR⁸ and —NR⁸R⁹;
R² is selected from the group consisting of H, C₁-C₆ alkyl, C₂-C₆ alkenyl, C₂-C₆ alkyl, C₃-C₆ cycloalkyl, C₄-C₇ cycloalkylalkyl and C₁-C₆ haloalkyl;
R³ is selected from the group consisting of H, halogen, C₁-C₆ alkyl, C₁-C₆ haloalkyl and C₃-C₆ cycloalkyl, wherein C₁-C₆ alkyl, C₁-C₆ haloalkyl and C₃-C₆ cycloalkyl are optionally substituted with 1 to 3 substituents selected independently at each occurrence from OR⁸ and NR⁸R⁹;
R⁴, R⁵, and R⁶ are each independently selected at each occurrence thereof from the group consisting of H, halogen, —OR¹⁰, —NO₂, —NR¹⁰R¹¹, —NR¹⁰C(O)R¹¹, —NR¹⁰C(O)NR¹¹R¹², —S(O)ₙR¹¹, —CN, —C(O)R¹¹, —C(O)₂R¹¹, —C(O)NR¹¹R¹² C₁-C₆ alkyl C₂-C₆ alkenyl, C₂-C₆ alkynyl, C₃-C₆ cycloalkyl and C₄-C₇ cycloalkylalkyl, wherein each of C₁-C₆ alkyl, C₂-C₆ alkenyl, C₂-C₆ alkynyl, C₃-C₆ cycloalkyl and C₄-C₇ cycloalkylalkyl are optionally substituted with 1 to 3 substituents independently selected at each occurrence with from C₁-C₃ alkyl, halogen, ═O, —CN, —OR⁸, —NR⁸R⁹ and phenyl, and wherein phenyl is optionally substituted 1-3 substituents selected independently at each occurrence from halogen, —CN, C₁-C₄ alkyl, C₁-C₄ haloalkyl, —OR⁸ and —NR⁸R⁹;
alternatively R¹ and R⁶ taken together are —O—C(R¹¹)₂—O—;
R⁷ is selected from the group consisting of H, halogen and OR¹⁰;
R⁸ and R⁹ are each independently selected from the group consisting of H, C₁-C₄ alkyl, C₁-C₄ haloalkyl, C₁-C₄ alkoxyalkyl, C₁-C₄ alkoxyalkylalkyl, C₃-C₆ cycloalkyl, C₄-C₇ cyclooalkylalkyl, —C(O)R¹², phenyl and benzyl, wherein phenyl and benzyl are optionally substituted with 1 to 3 substituents selected independently at each occurrence from halogen, cyano, C₁-C₄ alkyl, C₁-C₄ haloalkyl, C₁-C₄ alkoxy and C₁-C₄ haloalkoxy, or R⁸ and R⁹ are taken together with the nitrogen to which they are attached to form a piperidine, pyrrolidine, piperazine, N-methylpiperazine, morpholine, or thiomorpholine ring;
R¹⁰ is selected from the group consisting of H, C₁-C₄ alkyl, C₁-C₄ haloalkyl, C₁-C₄ alkoxyalkyl, C₃-C₆ cycloalkyl, C₄-C₇ cycloalkylalkyl, —C(O)R¹², phenyl and benzyl, wherein phenyl and benzyl are optionally substituted with 1 to 3 substituents selected independently at each occurrence from halogen, —NH₂, —OH, cyano, C₁-C₄ alkyl, C₁-C₄ haloalkyl, C₁-C₄ alkoxy and C₁-C₄ haloalkoxy;
R¹¹ is selected from the group consisting of H, C₁-C₄ alkyl, C₁-C₄ haloalkyl, C₁-C₄ alkoxyalkyl, C₃-C₆ cycloalkyl, C₄-C₇ cycloalkylalkyl, phenyl and benzyl, where phenyl and benzyl are optionally substituted with 1 to 3 substituents selected independently at each occurrence from halogen, —NH₂, —OH, cyano, C₁-C₄ alkyl, C₁-C₄ haloalkyl, C₁-C₄ alkoxy and C₁-C₄ haloalkoxy, or R¹⁰ and R¹¹ are taken together with the nitrogen to which they are attached to form a piperidine, pyrrolidine, N-methylpiperazine, morpholine, or thiomorpholine ring, with the proviso that only one of R⁸ and R9 or R¹⁰ and R¹¹ are taken together with the nitrogen to which they are attached to form a piperidine, pyrrolidine, piperaine, N-methylpiperazine, morpholine, or thiomorpholine ring;
R¹² is selected from the group consisting of C₁-C₄ alkyl, C₁-C₄ haloalkyl and phenyl;
X is selected from the group consisting of O, NR¹³ and S;
the ring containing X is selected from furan, pyrrole, thiophene, dihydrofuran, dihydropyrrole, and dihydrothiophene; n is 0, 1, or 2; and,
R¹³ is selected from the group consisting of H, C₁-C₆ alkyl, benzyl and phenyl, wherein C₁-C₆ alkyl, benzyl and phenyl are optionally substituted with 1-3 substituents independently at each occurrence from halogen, —NH₂, —OH, cyano, C₁-C₄ alkyl, C₁-C₄ haloalkyl, C₁-C₄ alkoxy and C₁-C₄ haloalkoxy,
or a pharmaceutically acceptable salt thereof or an isomer or prodrug thereof to a patient in need thereof.

2. A method of claim 1, wherein R¹ is C₁-C₆ alkyl.

3. A method of claim 2, wherein R¹ is CH₃.

4. A method of claim 1, wherein R² is H, C₁-C₆ alkyl, C₃-C₆ cycloalkyl, or C₁-C₆ haloalkyl.

5. A method of claim 4, wherein R² is H or C₁-C₆ alkyl.

6. A method of claim 5, wherein R² is H.

7. A method of claim 1, wherein R³ is at each occurrence thereof independently H, halogen, C₁-C₆ alkyl, or C₁-C₆ alkyl substituted with from 1 to 3 of OR⁸ or NR⁸R⁹.

8. A method of claim 7, wherein R³ is H or C₁-C₆ alkyl.

9. A method of claim 8, wherein R³ is H.

10. A method of claim 1, wherein R¹ is CH₃, R² is H and R³ is H.

11. A method of claim 1, wherein R⁴, R⁵ and R⁶ are each independently H, halogen, C₁-C₆ alkyl or —OR¹⁰.

12. A method of claim 11, wherein at least one of R⁴, R⁵ and R⁶ is H.

13. A method of claim 12, wherein each of R⁴, R⁵ and R⁶ are H.

14. A method of claim 12, wherein one of R⁴, R⁵ and R⁶ is halogen.

15. A method of claim 1, wherein R¹ is CH₃, R² and R³ are each H, and at least one of R⁴, R⁵, and R⁶ is H.

16. A method of claim 1 wherein the compound is a compound of Formula (10): 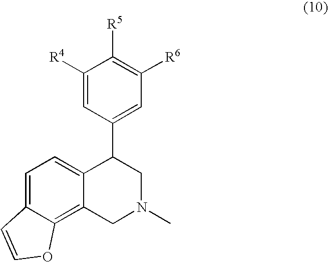
or a pharmaceutically acceptable salt form thereof selected from the group consisting essentially of
a compound of Formula (10) wherein R⁴ is H, R⁵ is H and R⁶ is H;
a compound of Formula (10) wherein R⁴ is H, R⁵ is Me and R⁶ is H;
a compound of Formula (10) wherein R⁴ is Cl, R⁵ is H and R⁶ is H; and
a compound of Formula (10) wherein R⁴ is H, R⁵ is F and R⁶ is H.

17. A method of claim 1 wherein the compound is a compound of Formula (20): 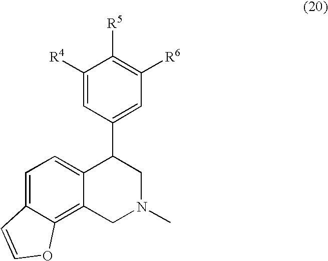
or a pharmaceutically acceptable salt form thereof selected from the group consisting essentially of:
a compound of Formula (20) wherein R⁴ is H, R⁵ is H and R⁶ is H;
a compound of Formula (20) wherein R⁴ is H, R⁵ is Me and R⁶ is H;
a compound of Formula (20) wherein R⁴ is H, R⁵ is Cl and R⁶ is H;
a compound of Formula (20) wherein R⁴ is H, R⁵ is F and R⁶ is H; and
a compound of Formula (20) wherein R⁴ is F, R⁵ is H and R⁶ is F.

18. A method of claim 1 wherein the compound is a compound of Formula (30): 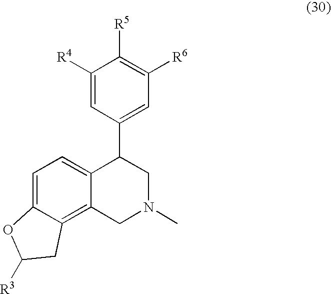
or a pharmaceutically acceptable salt form thereof selected from the group consisting essentially of
a compound of Formula (30) wherein R³ is H, R⁴ is H, R⁵ is H and R⁶ is H;
a compound of Formula (30) wherein R³ is H, R⁴ is F, R⁵ is F and R⁶ is H;
a compound of Formula (30) wherein R³ is H, R⁴ is F, R⁵ is H and R⁶ is F;
a compound of Formula (30) wherein R³ is H, R⁴ is H, R⁵ is F and R⁶ is H;
a compound of Formula (30) wherein R³ is H, R⁴ is Cl, R⁵ is H and R⁶ is H;
a compound of Formula (30) wherein R³ is H, R⁴ is H, R⁵ is Cl and R⁶ is H;
a compound of Formula (30) wherein R³ is H, R⁴ is H, R⁵ is Cl and R⁶ is F;
a compound of Formula (30) wherein R³ is H, R⁴ is H, R⁵ is F and R⁶ is Cl;
a compound of Formula (30) wherein R³ is H, R⁴ is F, R⁵ is H and R⁶ is Cl;
a compound of Formula (30) wherein R³ is H, R⁴ is H, R⁵ is OMe and R⁶ is H; and
a compound of Formula (30) wherein R³ is H, R⁴ is F, R⁵ is H and R⁶ is H.

19. A method of claim 1 wherein the compound is a compound of Formula (40): 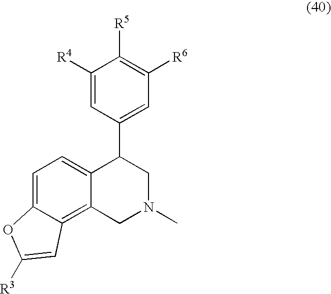
or a pharmaceutically acceptable salt form thereof selected from the group consisting essentially of
a compound of Formula (40) wherein R³ is H, R⁴ is H, R⁵ is H and R⁶ is H;
a compound of Formula (40) wherein R³ is H, R⁴ is F, R⁵ is F and R⁶ is H;
a compound of Formula (40) wherein R³ is H, R⁴ is F, R⁵ is H and R⁶ is F;
a compound of Formula (40) wherein R³ is H, R⁴ is F, R⁵ is H and R⁶ is H;
a compound of Formula (40) wherein R³ is H, R⁴ is H, R is F and R⁶ is H;
a compound of Formula (40) wherein R³ is H, R⁴ is Cl, R⁵ is H and R⁶ is H;
a compound of Formula (40) wherein R³ is H, R⁴ is H, R⁵ is Cl and R⁶ is H;
a compound of Formula (40) wherein R³ is H, R⁴ is H, R⁵ is Cl and R⁶ is F;
a compound of Formula (40) wherein R³ is H, R⁴ is H, R⁵ is F and R⁶ is Cl;
a compound of Formula (40) wherein R³ is H, R⁴ is F, R⁵ is H and R⁶ is Cl;
a compound of Formula (40) wherein R³ is H, R⁴ is H, R⁵ is OMe and R⁶ is H;
a compound of Formula (40) wherein R³ is Me, R⁴ is H, R⁵ is H and R⁶ is H;
a compound of Formula (40) wherein R³ is Et, R⁴ is H, R⁵ is H and R⁶ is H; and
a compound of Formula (40) wherein R³ is CH₂OH, R⁴ is H, R is H and R⁶ is H.

20. A method of claim 1 wherein the compound is a compound of Formula (50): 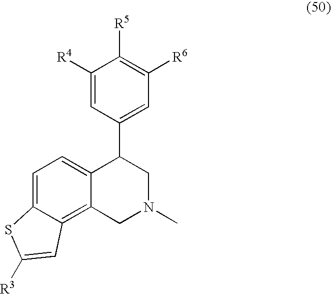
or a pharmaceutically acceptable salt form thereof selected from the group consisting essentially of:
a compound of Formula (50) wherein R³ is H, R⁴ is H, R⁵ is H and R⁶ is H.

21. A method of claim 1 wherein the compound is a compound of Formula (60): 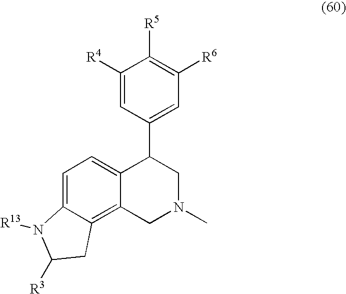
or a pharmaceutically acceptable salt form thereof selected from the group consisting essentially of
a compound of Formula (60) wherein R³ is H, R⁴ is H, R⁵ is H and R⁶ is H and R¹³ is H;
a compound of Formula (60) wherein R³ is H, R⁴ is H, R⁵ is H, R⁶ is H and R¹³ is Me;
a compound of Formula (60) wherein R³ is H, R⁴ is H, R⁵ is H, R⁶ is H and R¹³ is Et;
a compound of Formula (60) wherein R³ is H, R⁴ is H, R⁵ is F, R⁶ is F and R¹³ is H;
a compound of Formula (60) wherein R³ is H, R⁴ is H, R⁵ is F, R⁶ is F and R¹³ is Me;
a compound of Formula (60) wherein R³ is H, R⁴ is F, R⁵ is H, R⁶ is F and R¹³ is H;
a compound of Formula (60) wherein R³ is H, R⁴ is F, R⁵ is H, R⁶ is F and R¹³ is Me;
a compound of Formula (60) wherein R³ is H, R⁴ is Cl, R⁵ is H, R⁶ is H and R¹³ is H;
a compound of Formula (60) wherein R³ is H, R⁴ is Cl, R⁵ is H, R⁶ is H and R¹³ is Me;
a compound of Formula (60) wherein R³ is H, R⁴ is F, R is H, R⁶ is H and R¹³ is H;
a compound of Formula (60) wherein R³ is H, R⁴ is H, R⁵ is F, R⁶ is H and R¹³ is H;
a compound of Formula (60) wherein R³ is H, R⁴ is F, R⁵ is Cl, R⁶ is H and R¹³ is H;
a compound of Formula (60) wherein R³ is H, R⁴ is F, R⁵ is Cl, R⁶ is H and R¹³ is Me;
a compound of Formula (60) wherein R³ is H, R⁴ is Cl, R⁵ is F, R⁶ is H and R¹³ is H; and
a compound of Formula (60) wherein R³ is H, R⁴ is Cl, R⁵ is F, R⁶ is H and R¹³ is Me.

22. A method of claim 1 wherein the compound is a compound of Formula (70): 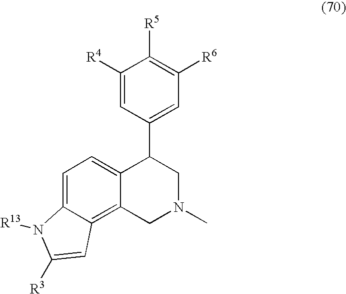
or a pharmaceutically acceptable salt form thereof selected from the group consisting essentially of
a compound of Formula (70) wherein R³ is H, R⁴ is H, R is H, R⁶ is H and R¹³ is H;
a compound of Formula (70) wherein R³ is H, R⁴ is H, R⁵ is H, R⁶ is H and R¹³ is Me;
a compound of Formula (70) wherein R³ is H, R⁴ is H, R⁵ is H, R⁶ is H and R¹³ is Et;
a compound of Formula (70) wherein R³ is H, R⁴ is H, R⁵ is H, R⁶ is H and R¹³ is Bn;
a compound of Formula (70) wherein R³ is H, R⁴ is H, R⁵ is F, R⁶ is F and R¹³ is H;
a compound of Formula (70) wherein R³ is H, R⁴ is H, R⁵ is F, R⁶ is F and R¹³ is Me;
a compound of Formula (70) wherein R³ is H, R⁴ is F, R⁵ is H, R⁶ is F and R¹³ is Me;
a compound of Formula (70) wherein R³ is H, R⁴ is Cl, R is H, R⁶ is H and R¹³ is H;
a compound of Formula (70) wherein R³ is H, R⁴ is Cl, R⁵ is H, R⁶ is H and R¹³ is Me;
a compound of Formula (70) wherein R³ is H, R⁴ is F, R⁵ is H, R⁶ is H and R¹³ is H;
a compound of Formula (70) wherein R³ is H, R⁴ is F, R⁵ is H, R⁶ is H and R¹³ is Me;
a compound of Formula (70) wherein R³ is H, R⁴ is H, R⁵ is F, R⁶ is H and R¹³ is H;
a compound of Formula (70) wherein R³ is H, R⁴ is F, R⁵ is Cl, R⁶ is H and R¹³ is H;
a compound of Formula (70) wherein R³ is H, R⁴ is F, R is Cl, R⁶ is H and R¹³ is Me;
a compound of Formula (70) wherein R³ is H, R⁴ is Cl, R is F, R⁶ is H and R¹³ is H; and
a compound of Formula (70) wherein R³ is H, R⁴ is Cl, R⁵ is F, R⁶ is H and R¹³ is Me.

23. A method of claim 1 wherein the compound is a compound of Formula (80): 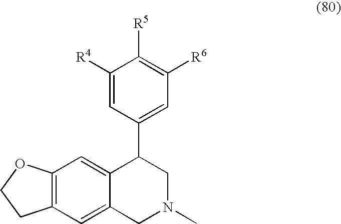
or a pharmaceutically acceptable salt form thereof selected from the group consisting essentially of
a compound of Formula (80) wherein R⁴ is H, R⁵ is H and R⁶ is H;
a compound of Formula (80) wherein R⁴ is H, R⁵ is F and R⁶ is H; and
a compound of Formula (80) wherein R⁴ is H, R⁵ is F and R⁶ is F.

24. A method of claim 1 wherein the compound is a compound of Formula (90): 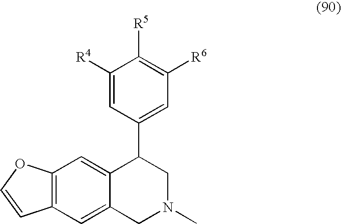
or a pharmaceutically acceptable salt form thereof selected from the group consisting essentially of
a compound of Formula (90) wherein R⁴ is H, R⁵ is H and R⁶ is H;
a compound of Formula (90) wherein R⁴ is H, R⁵ is F and R⁶ is F; and
a compound of Formula (90) wherein R⁴ is H, R⁵ is F and R⁶ is H.

25. A method of claim 1 wherein the compound is a compound of Formula (100): 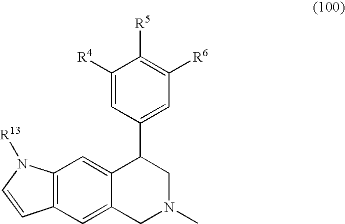
or a pharmaceutically acceptable salt form thereof selected from the group consisting essentially of:
a compound of Formula (100) wherein R⁴ is H, R is H, R⁶ is H and R¹³ is H.

26. A method of claim 1 wherein the compound is a compound of Formula (110): 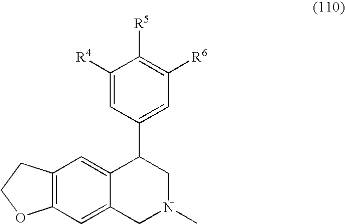
or a pharmaceutically acceptable salt form thereof selected from the group consisting essentially of:
a compound of Formula (110) wherein R⁴ is H, R⁵ is H and R⁶ is H;
a compound of Formula (110) wherein R⁴ is H, R⁵ is F and R⁶ is F;
a compound of Formula (110) wherein R⁴ is H, R⁵ is F and R⁶ is H;
a compound of Formula (110) wherein R⁴ is H, R⁵ is H and R⁶ is Cl;
a compound of Formula (110) wherein R⁴ is H, R⁵ is Cl and R⁶ is F;
a compound of Formula (110) wherein R⁴ is H, R⁵ is F and R⁶ is Cl; and
a compound of Formula (110) wherein R⁴ is H, R⁵ is OMe and R⁶ is H.

27. A method of claim 1 wherein the compound is a compound of Formula (120): 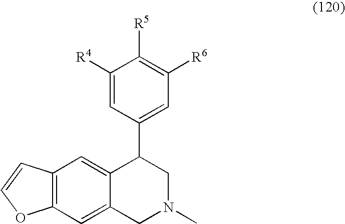
or a pharmaceutically acceptable salt form thereof selected from the group consisting essentially of
a compound of Formula (120) wherein R⁴ is H, R⁵ is H and R⁶ is H;
a compound of Formula (120) wherein R⁴ is H, R is F and R⁶ is F;
a compound of Formula (120) wherein R⁴ is H, R⁵ is F and R⁶ is H;
a compound of Formula (120) wherein R⁴ is H, R⁵ is H and R⁶ is Cl;
a compound of Formula (120) wherein R⁴ is H, R⁵ is Cl and R⁶ is F;
a compound of Formula (120) wherein R⁴ is H, R⁵ is OMe and R⁶ is H; and
a compound of Formula (120) wherein R⁴ is H, R⁵ is F and R⁶ is Cl.

28. A method of claim 1 wherein the compound is a compound of Formula (130): 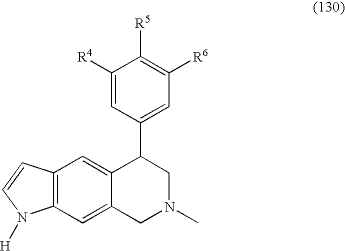
or a pharmaceutically acceptable salt form thereof selected from the group consisting essentially of
a compound of Formula (130) wherein R⁴ is H, R⁵ is H and R⁶ is H; and
a compound of Formula (130) wherein R⁴ is H, R⁵ is Bn and R⁶ is H.

29. A method of claim 1 wherein the compound is a compound of Formula (140): 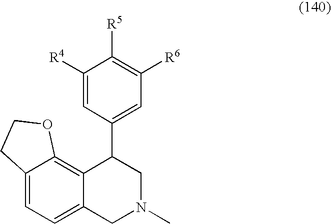
or a pharmaceutically acceptable salt form thereof selected from the group consisting essentially of
a compound of Formula (140) wherein R⁴ is H, R⁵ is H and R⁶ is H;
a compound of Formula (140) wherein R⁴ is H, R⁵ is F and R⁶ is H;
a compound of Formula (140) wherein R⁴ is H, R⁵ is F and R⁶ is Cl;
a compound of Formula (140) wherein R⁴ is H, R⁵ is Cl and R⁶ is F;
a compound of Formula (140) wherein R⁴ is H, R⁵ is H and R⁶ is Cl;
a compound of Formula (140) wherein R⁴ is H, R⁵ is OMe and R⁶ is H;
a compound of Formula (140) wherein R⁴ is H, R⁵ is F and R⁶ is F.

30. A method of claim 1 wherein the compound is a compound of Formula (150): 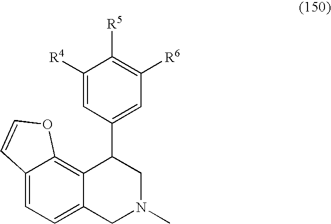
or a pharmaceutically acceptable salt form thereof selected from the group consisting essentially of
a compound of Formula (150) wherein R⁴ is H, R⁵ is H and R⁶ is H;
a compound of Formula (150) wherein R⁴ is H, R⁵ is F and R⁶ is H;
a compound of Formula (150) wherein R⁴ is H, R⁵ is F and R⁶ is Cl;
a compound of Formula (150) wherein R⁴ is H, R⁵ is Cl and R⁶ is F;
a compound of Formula (150) wherein R⁴ is H, R⁵ is H and R⁶ is Cl;
a compound of Formula (150) wherein R⁴ is H, R⁵ is OMe and R⁶ is H; and
a compound of Formula (150) wherein R⁴ is H, R⁵ is F and R⁶ is F.

31. A method of claim 1 wherein the compound is a compound of Formula (160): 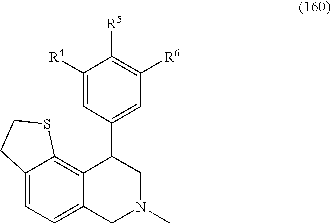
or a pharmaceutically acceptable salt form thereof selected from the group consisting essentially of:
a compound of Formula (160) wherein R⁴ is H, R⁵ is H and R⁶ is H.

32. A method of claim 1 wherein the compound is a compound of Formula (170): 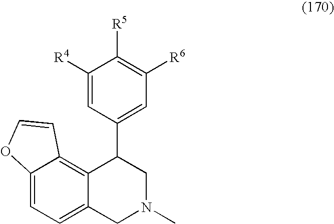
or a pharmaceutically acceptable salt form thereof selected from the group consisting essentially of:
a compound of Formula (170) wherein R⁴ is H, R⁵ is H and R⁶ is H;
a compound of Formula (170) wherein R⁴ is H, R⁵ is F and R⁶ is H; and
a compound of Formula (170) wherein R⁴ is H, R⁵ is F and R⁶ is F.

33. A method of claim 1 wherein the compound is a compound of Formula (180): 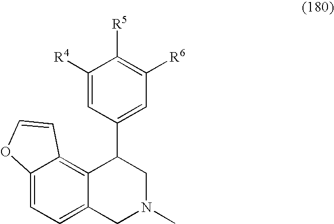
or a pharmaceutically acceptable salt form thereof selected from the group consisting essentially of:
a compound of Formula (180) wherein R⁴ is H, R⁵ is H and R⁶ is H;
a compound of Formula (180) wherein R⁴ is H, R⁵ is F and R⁶ is H; and
a compound of Formula (180) wherein R⁴ is H, R⁵ is F and R⁶ is F.

34. A method of claim 1 wherein the compound is a compound of Formula (190): 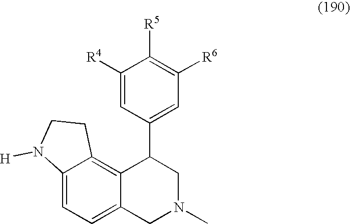
or a pharmaceutically acceptable salt form thereof selected from the group consisting essentially of:
a compound of Formula (190) wherein R⁴ is H, R⁵ is H and R⁶ is H.

35. A method of claim 1 wherein the compound is a compound of Formula (200): 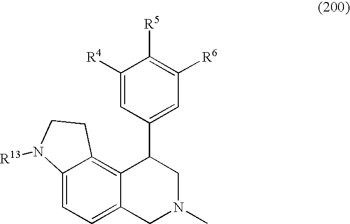
or a pharmaceutically acceptable salt form thereof selected from the group consisting essentially of
a compound of Formula (200) wherein R⁴ is H, R⁵ is H, R⁶ is H and R¹³ is H; and
a compound of Formula (200) wherein R⁴ is H, R⁵ is H, R⁶ is H and R¹³ is Me.

36. A method of claim 1 wherein the compound is selected from the group consisting of:
(R)-2-methyl-4-phenyl-1,2,3,4,8,9-hexahydro-furo[2,3-h]isoquinoline;
(S)-2-methyl-4-phenyl-1,2,3,4, 8,9-hexahydro-furo[2,3-h]isoquinoline;
(R)-7-methyl-5-phenyl-5,6,7,8-tetrahydro-furo[3,2-g]isoquinoline;
(S)-7-methyl-5-phenyl-5,6,7,8-tetrahydro-furo[3,2-g]isoquinoline;
(R)-4-(4-fluoro-phenyl)-2-methyl-1,2,3,4-tetrahydrofuro[2,3-h]isoquinoline;
(S)-4-(4-fluoro-phenyl)-2-methyl-1,2,3,4-tetrahydrofuro[2,3-h]isoquinoline;
(R)-4-(3,4-difluoro-phenyl)-2-methyl-1,2,3,4-tetrahydrofuro[2, 3-h]isoquinoline;
(S)-4-(3,4-difluoro-phenyl)-2-methyl-1,2,3,4-tetrahydrofuro[2,3-h]isoquinoline;
(R)-2-methyl-4-phenyl-1,2,3,4-tetrahydro-furo[2,3-h]isoquinoline;
(S)-2-methyl-4-phenyl-1,2,3,4-tetrahydro-furo[2,3-h]isoquinoline;
(R)-4-(4-chloro-phenyl)-2-methyl-1,2,3,4-tetrahydro-furo[2,3-h]isoquinoline;
(S)-4-(4-chloro-phenyl)-2-methyl-1,2,3,4-tetrahydrofuro[2,3-h]isoquinoline;
(R)-8-methyl-6-phenyl-2,3,6,7,8,9-hexahydro-furo[3,2-h]isoquinoline;
(S)-8-methyl-6-phenyl-2,3,6,7,8,9-hexahydro-furo[3,2-h]isoquinoline;
(R)-4-(4-fluoro-phenyl)-2-methyl-1,2,3,4-tetrahydrofuro[2,3-h]isoquinoline;
(S)-4-(4-fluoro-phenyl)-2-methyl-1,2,3,4-tetrahydro-furo[2,3-h]isoquinoline;
(R)-4-(3,5-difluoro-phenyl)-2-methyl-1,2,3,4-tetrahydrofuro[2,3-h]isoquinoline;
(S)-4-(3,5-difluoro-phenyl)-2-methyl-1,2,3,4-tetrahydro-furo[2,3-h]isoquinoline;
(R)-2-methyl-4-phenyl2,3,4,7-tetrahydro-1H-pyrrolo[2,3-h]isoquinoline; and
(S)-2-methyl-4-phenyl-2,3,4,7-tetrahydro-1H-pyrrolo[2,3-h]isoquinoline.

37. A method of claim 1 wherein the compound is selected from the group consisting of
(+)-2-methyl-4-phenyl-1,2,3,4,8,9-hexahydro-furo[2,3-h]isoquinoline;
(−)-2-methyl-4-phenyl-1,2,3,4,8,9-hexahydro-furo[2,3-h]isoquinoline;
(+)-7-methyl-5-phenyl-5,6,7,8-tetrahydro-furo[3,2-g]isoquinoline;
(−)-7-methyl-5-phenyl-5,6,7,8-tetrahydro-furo[3,2-g]isoquinoline;
(+)-4-(4-fluoro-phenyl)-2-methyl-1,2,3,4-tetrahydro-furo[2,3-h]isoquinoline;
(−)-4-(4-fluoro-phenyl)-2-methyl-1,2,3,4-tetrahydro-furo[2,3-h]isoquinoline;
(+)-4-(3,4-difluoro-phenyl)-2-methyl-1,2,3,4-tetrahydro-furo[2,3-h]isoquinoline;
(−)-4-(3,4-difluoro-phenyl)-2-methyl-1,2,3,4-tetrahydro-furo[2,3-h]isoquinoline;
(+)-2-methyl-4-phenyl-1,2,3,4-tetrahydro-furo[2,3-h]isoquinoline;
(−)-2-methyl-4-phenyl-1,2,3,4-tetrahydro-furo[2,3-h]isoquinoline;
(+)-4-(4-chloro-phenyl)-2-methyl-1,2,3,4-tetrahydrofuro[2,3-h]isoquinoline;
(−)-4-(4-chloro-phenyl)-2-methyl-1,2,3,4-tetrahydro-furo[2,3-h]isoquinoline;
(+)-8-methyl-6-phenyl-2,3,6,7,8,9-hexahydro-furo[3,2h]isoquinoline;
(−)-8-methyl-6-phenyl-2,3,6,7,8,9-hexahydro-furo[3,2-h]isoquinoline;
(+)-4-(4-fluoro-phenyl)-2-methyl-1,2,3,4-tetrahydro-furo[2,3-h]isoquinoline;
(−)-4-(4-fluoro-phenyl)-2-methyl-1,2,3,4-tetrahydrofuro[2,3-h]isoquinoline;
(+)-4-(3,5-difluoro-phenyl)-2-methyl-1,2,3,4-tetrahydro-furo[2,3-h]isoquinoline;
(−)-4-(3,5-difluoro-phenyl)-2-methyl-1,2,3,4-tetrahydro-furo[2,3-h]isoquinoline;
(+)-2-methyl-4-phenyl-2,3,4,7-tetrahydro-1H-pyrrolo[2,3-h]isoquinoline; and
(−)-2-methyl-4-phenyl-2,3,4,7-tetrahydro-1H-pyrrolo[2,3-h]isoquinoline.
